(19) United States
(12) Patent Application Publication
(10) Pub. No.: US 2023/0087487 A1
(43) Pub. Date: Mar. 23, 2023

(54) TOPICAL DELIVERY OF RNA INTERFERENCE AGENTS USING IONIC LIQUID

(71) Applicant: CAGE BIO, INC., San Diego, CA (US)

(72) Inventors: Nitin JOSHI, San Diego, CA (US); Kevin GELSTON, San Diego, CA (US); Marina SHEVACHMAN, San Diego, CA (US)

(21) Appl. No.: 17/784,053

(22) PCT Filed: Dec. 9, 2020
(86) PCT No.: PCT/US20/64113

§ 371 Date: Jun. 9, 2022

Related U.S. Application Data
(60) Provisional application No. 62/945,726, filed on Dec. 9, 2019.

(51) Int. Cl. A61K 9/00; C12N 15/113; A61K 9/06; A61K 47/54

(52) U.S. Cl. CPC A61K 9/0014; C12N 15/113; A61K 9/06; A61K 47/542; C12N 2310/14; C12N 2320/32

(57) ABSTRACT

One aspect of the present disclosure is directed to a method of delivering the RNAi agents to or through a skin. Another aspect of the present disclosure is directed to a method of reducing expression or translation of a target gene within a stratum corneum layer, an epidermis layer, a dermis layer, a subcutaneous tissue layer, or a muscle tissue. Another aspect of the present disclosure is directed to a topical composition, comprising the RNAi agents or a pharmaceutically acceptable salt thereof, and an ionic liquid in an amount sufficient to allow a therapeutically effective amount of the RNAi agents to reach a target depth within or beyond the skin.

### BACKGROUND OF THE DISCLOSURE

RNA interference-based therapeutics are useful in treating a variety of diseases and conditions. Examples of RNA interference agents are microRNAs (miRNAs), small interfering RNAs (siRNAs), and short hairpin RNAs (shRNAs). Sonic RNA interference agents can direct enzyme complexes to degrade messenger RNA (mRNA) agents and thus decrease their activity by decreasing translation, via post-transcriptional gene silencing. Furthermore, transcription can be disrupted via the pre-transcriptional silencing mechanism of RNA interference.

### SUMMARY OF THE DISCLOSURE

Disclosed herein, in certain embodiments, is a topical composition, comprising the RNAi agents or a pharmaceutically acceptable salt thereof, and an ionic liquid in an amount sufficient to allow a therapeutically effective amount of the RNAi agents to reach a target depth within or beyond the skin, wherein the ionic liquid comprises a choline cation and a fatty acid anion.

In sonic embodiments, the RNAi agents are selected from the group consisting of interfering RNAs (siRNAs), microRNAs (miRNAs), small hairpin RNAs (shRNAs), and combinations thereof.

In some embodiments, the RNAi agents are selected from the group consisting of siRNA, miRNAs, and combinations thereof.

In some embodiments, the RNAi agents are siRNAs.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, meristic acid, pentadecylic acid, palmitic acid, tnargaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

In some embodiments, the fatty acid is selected from the group consisting oleic acid, geranic acid, hexanoic acid, and malonic acid.

In some embodiments, the fatty acid is geranic acid.

In some embodiments, the ionic liquid is a deep eutectic solvent (DES).

In some embodiments, the ionic liquid comprises the choline cation and fatty acid anion in a molar ratio in a range of 1:1 to 1:4 of choline cation to fatty acid anion.

In some embodiments, the ionic liquid comprises the choline cation and fatty acid anion in a molar ratio of 1:2 of choline cation to fatty acid anion.

In some embodiments, the pharmaceutical composition consists essentially of the RNAi agents and the ionic liquid.

In sonic embodiments, the pharmaceutical composition further comprises a pharmaceutically acceptable carrier.

In some embodiments, the pharmaceutically acceptable carrier is an aqueous carrier.

In some embodiments, the pharmaceutically acceptable carrier comprises an ointment base.

In some embodiments, the ionic liquid is present in the pharmaceutical composition at a concentration of from about 1% to about 99%.

In sonic embodiments, the ionic liquid is present in the pharmaceutical composition at a concentration of from about 5% to about 85%.

In some embodiments, the ionic liquid is present in the pharmaceutical composition at a concentration of from about 10% to about 90%.

In some embodiments, the ionic liquid is present in the pharmaceutical composition at a concentration of from about 20% to about 80%.

In some embodiments, the ionic liquid is present in the pharmaceutical composition at a concentration of from about 30% to about 60%.

In some embodiments, the ionic liquid is present in the pharmaceutical composition at a concentration of from about 40% to about 60%.

In some embodiments, the ionic liquid is present in the pharmaceutical composition at a concentration of about 45% to about 55%.

In some embodiments, the ionic liquid is present in the pharmaceutical composition at a concentration of about 50%.

In sonic embodiments, the pharmaceutical composition is essentially free of any additional penetration enhancer.

In some embodiments, the target depth is from about 0.05 mm to about 20 mm. In some embodiments, the target depth is from about 1 mm to about 10 mm.

In some embodiments, the pharmaceutical composition allows therapeutically effective amount of the RNAi agents to be delivered into the stratum corneum layer. In some embodiments, the total amount of the RNAi agents delivered into the stratum corneum layer provided by the pharmaceutical composition is greater than the total amount of the RNAi agents delivered into a stratum corneum layer provided by a control composition without the ionic liquid.

In some embodiments, the pharmaceutical composition allows therapeutically effective amount of the RNAi agents to be delivered into at least one of epidermis and dermis layer. In some embodiments, the total amount of the RNAi agents delivered to the at least one of epidermis and dermis layers provided by the pharmaceutical composition is more than the total amount of the RNAi agents delivered into at least one of epidermis and dermis layers provided by a control composition without the ionic liquid.

In some embodiments, the pharmaceutical composition allows therapeutically effective amount of the RNAi agents to reach beyond a dermis layer. In sonic embodiments, the total amount of the RNAi agents beyond the dermis layer provided by the pharmaceutical composition is more than the total amount of the RNAi. agents delivered beyond a dermis layer provided by a control composition without the ionic liquid.

Disclosed herein, in certain embodiments, is a method of delivering RNA interference (RNAi) agents to or through a skin, using the topical compositions disclosed herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows delivery of a non-limiting topical compositions comprising an siRNA and ionic liquid according to some embodiments of the present disclosure, in comparison with a control composition of the siRNA in RNAse-free water without the ionic liquid, showing delivery of the siRNA to stratum corneum, epidermis, dermis, and beyond the skin layers.

### DETAILED DESCRIPTION OF THE DISCLOSURE

Disclosed herein, in certain embodiments, is a topical composition, comprising the RNAi agents or a pharmaceutically acceptable salt thereof, and an ionic liquid in an amount sufficient to allow a therapeutically effective amount of the RNAi agents to reach a target depth within or beyond the skin, wherein the ionic liquid comprises a choline cation and a fatty acid anion. RNAi agents

RNA interference (RNAi) is the process by which the expression of a target gene is effectively silenced or knocked down by the selective inactivation of its corresponding mRNA by double-stranded RNA (dsRNA). RNAi is activated by dsRNA species delivered to the cytoplasm of cells. The silencing mechanisms can either lead to the degradation of a target mRNA, as induced by small interfering RNAs (siRNAs) or short hairpin RNAs (shRNAs), or the suppression of translation of specific mRNAs, as induced by microRNA (miRNA). The focus of this review will be how shRNAs and siRNAs lead to protein knockdown. Through the activity of several proteins (discussed below), targeting of a cellular mRNA by short, anti-sense nucleic acids (siRNAs and shRNAs) results in its subsequent degradation. This, in turn, blocks further expression/accumulation of the proteins, leading to a decrease in its levels, and eventual knockdown.

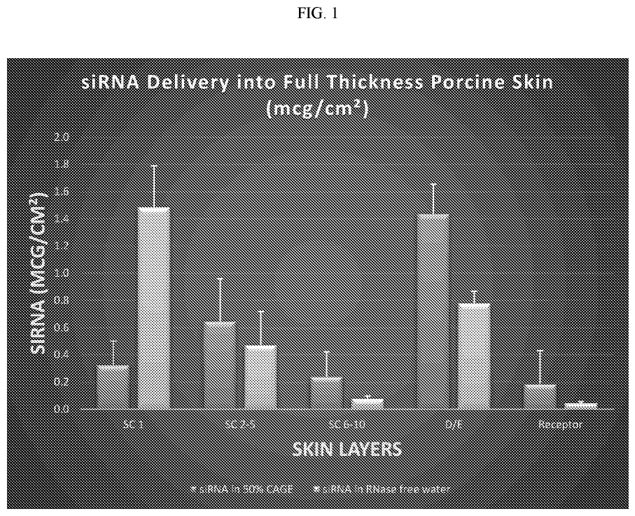

Two key approaches to RNAi that have gained substantial interest for use in gene silencing are the double-stranded small interfering RNAs (siRNAs) and the vector-based short hairpin RNAs (shRNAs). While both siRNAs and shRNAs (FIG. 1) can be used for protein knockdown, there are differences in their mechanisms of action (FIG. 2). Either long dsRNAs or short duplexes of about 21 base pairs (bps) can be introduced directly into cells in tissue culture (see Mechanisms of Delivery for further details). While there are a few reports of siRNAs being translocated to the nucleus upon transfection into cells, it is inure generally accepted that they accumulate in the cytoplasm. Upon introduction to the cell, the long dsRNAs form a complex with Dicer, a dsRNA-specific RNase III enzyme that processes them into 21-23 nucleotide (nt) siRNAs with characteristic 2 nt 3′ overhangs. The cleaved products arc then incorporated into the RISC, which is composed of Argonaute-2 (Ago-2), Dicer, and TAR-RNA-binding protein (TRBP). The RNA duplex is separated, and one strand is removed from the complex. The strand with the lowest duplex stability at its 5′-end is selected for stable incorporation into the RISC.

shRNAs are synthesized in the nucleus of transfected/transduced cells and form hairpin structures that consist of a stein region of paired antisense and sense strands connected by unpaired nucleotides that make up a loop. They are converted into siRNAs by the same RNAi machinery that processes miRNAs. shRNAs are introduced into the nuclei of target cells using either bacterial or viral vectors that, in some cases, can stably integrate into the genome. shRNAs are transcribed by either RNA polymerase II or III, depending on the promoter driving their expression. These initial precursors are processed by Drosha and its dsRNA-binding partner DGCR8, resulting in species known as pre-shRNAs, before being exported to the cytoplasm by Exportin-5. The pre-shRNA is then cleaved by Dicer and TRBP/PACT, removing the hairpin and creating a 20-25 nt double-stranded siRNA with 2 nt 3′ overhangs at each end. This active siRNA is then loaded onto the RISC complex.

Once loaded onto the RISC, the process of target mRNA recognition and degradation by both shRNA and siRNA is similar. As a moiety of the RISC, the siRNA binds to the target mRNA in a sequence-specific manner that is mediated by complementary base pairing, leading to cleavage of the target RNA phosphate backbone near the center of the duplex via the action of the RNase-H like activity of Ago-2. An interesting feature of this system in some organisms is that annealing of the siRNA to the target nRNA allows the siRNA to act as a primer, while the target mRNA acts as a template for an RNA-dependent RNA-polymerase. This creates a new dsRNA, which is then processed by Dicer, creating a positive feedback loop that increases the pool of siRNAs.

Advantages of shRNA over siRNA include the ability to use viral vectors for delivery to overcome the difficulty of transfecting certain cell types, the option to control shRNA expression using inducible promoters, and the ability to co-express them with a reporter gene. Additionally, they may cause fewer off-target effects.

Similar to siRNAs, miRNAs silence gene expression post-transcriptionally, and are thought to regulate roughly 30% of human genes. miRNA precursors are naturally encoded in the genome, but miRNAs themselves can be artificially synthesized for therapeutic strategies. In the cell RNA polymerase II transcribes DNA into primary miRNA (pri-miRNA). The pri-miRNA forms a hairpin structure that is cleaved by Drosha, a ribonuclease with dsRNA specificity, into the precursor miRNA (pre-miRNA). The pre-miRNA is generally more than 100 nucleotides long and contains a hairpin loop and a double-strand region where the miRNA resides. Pre-miRNAs are transported by exportin 5 from the nucleus to the cytoplasm via the nuclear pore complex. Pre-miRNAs are then further processed by Drosha to release the hairpin. The RNAse RI enzyme Dicer then cleaves the loop of the hairpin in the pre-mRNAs generating the double-strand segment known as the miRNA. miRNA is then incorporated into the RISC complex by binding to AGO2. The guide strand is kept, and the other strand (“the passenger”) is degraded. Within the miRNA guide strand lies a seed sequence near the 5′-end; this seed sequence is composed of seven bases that are crucial for the complementary binding to the target mRNA. Sequences outside this seed region are less significant for binding. The guide strand of the miRNA leads the AGO2-RISC complex to the target mRNA. Generally, the complementary sequence lies in the 3′ untranslated region (UTR) of the mRNA, but it can also be found in the 5′ UTR or coding region. The mRNA is silenced by inhibition of translation and/or degradation by exonucleases.

Suitable viral vectors include recombinant retroviruses, lentiviruses, adenoviruses, adeno-associated viruses, and baculoviruses. These expression vectors are well known in the art (Boeckle and Wagner. The AAPS Journal. 8(4):E731-E742 (2006); Hu, Acta Pharmacologica Sinica, 26(4):405-416 (2005)). Bacterial expression vectors including plasmids, cosmids, phagemids, and equivalents thereof, are known in the art and discussed in detail in T. A. Brown. Chapter 2—Vectors for Gene Cloning: Plasmids and Bacteriophages. Gene Cloning and DNA Analysis: An Introduction (6th ed.), (2010) Wiley-Blackwell. ISBN 978-1405181730. Ionic liquids

Described herein, in certain embodiments, are compositions comprising an ionic liquid comprising a choline cation and a fatty acid anion. In some embodiments, the composition further comprises a pharmaceutically acceptable solvent. In some embodiments, the fatty acid is myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, enanthic acid, caprylic acid, pelargonic acid, cupric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid. In some embodiments, the fatty acid is geranic acid. In some embodiments, the fatty acid comprises 9 to 14 carbons, In some embodiments, the ionic liquid is liquid at room temperature. In some embodiments, the ionic liquid is liquid below 100° C.

In some embodiments, the ionic liquid is a deep eutectic solvent (DES). In some embodiments, a DES comprises excess carboxylate which precludes 1:1 ion pairing. In some embodiments, a DES further comprises a hydrogen-bond donor. In some embodiments, the hydrogen-bond donor is urea or citric acid. In some embodiments, the solvent properties of a DES are adjusted by changing the hydrogen-bond donor. In some embodiments, the ammonium salt of a DES interacts with a hydrogen-bond donor. In some embodiments, the DES has a melting point lower than either of the individual components (e.g. fatty acid and choline).

In some embodiments, the ionic liquid comprises a molar ratio of a choline cation to a fatty acid anion of 1:0.5 to 1:10. In some embodiments, the molar ratio of the choline cation to the fatty acid anion is about 1:0,5, 1:0.6, 1:0,7, 1:0.8, 1:0.9, 1:1.0; 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4,8,1:4.9. 1:5,0, 1:5.11, 1:5,2, 1:5.3, 1:5.4, 1:5.5, 1:5.6, 1:5,7,1:5.8, 1:5,9, 1:6.0, 1:6.1, 1:6.2, 1:6,3,1:6.4, 1:6.5, 1:6.6, 1:6,7, 1:6.8, 1:6.9, 1:7.0, 1:7.1, 1:7.2, 1:7.3, 1:7.4, 1:7.5, 1:7.6, 1:7.7, 1:7.8, 1:7.9, 1:8.0, 1:8.1, 1:8.2, 1:8.3, 1:8.4, 1:8.5, 1:8.6, 1:8.7, 1:8.8, 1:8.9, 1:9.0, 1:9.1, 1:9.2, 1:9.3, 1:9.4, 1:9.5, 1:9.6, 1:9.7, 1:9.8, 1:9.9, or about 1:10. In some embodiments, the molar ratio of the choline cation to the fatty acid anion is about. 1:1,1, 1:1.2, 1:1.3, 1:1.4, 1:1.5, 1:1.6, 1:1.7, 1:1.8, 1:1.9, or 1:2.0.

In sonic embodiments, the choline cation and fatty acid anion are in a molar ratio in the ionic liquid. In some embodiments, the choline cation and fatty acid anion are in a molar ratio of 1:1. In some embodiments, the term Composition B is used herein to refer to a composition or an ionic liquid comprising a 1:1 molar ratio of choline cation to geranic acid anion. In some embodiments, Composition B does not comprise water.

In other embodiments, the choline cation and fatty acid anion are in a molar ratio of 1:2. In some embodiments, the term Composition A is used herein to refer to a composition or an ionic liquid comprising a 1:2 molar ratio of choline cation to geranic acid anion. In some embodiments, Composition A does not comprise water.

In some embodiments, the chemical structure of choline is:

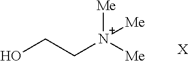

wherein X⁻ is a pharmaceutically acceptable anion.

In some embodiments, term choline refers to the class of quaternary ammonium salts containing the N,N,N-trimethylethanolammonium cation. In some embodiments, the X″ the right of the structure of choline denotes a pharmaceutically acceptable anion. In some embodiments the X⁻ is bicarbonate, carbonate, acetate, citrate, tartarate, bitartarate, lactate, chloride, bromide, or iodide. In some embodiments, the X⁻ is bicarbonate. In some embodiments, the choline is an anti-inflammatory agent.

In some embodiments, choline is in the form of a pharmaceutically acceptable salt. The type of pharmaceutical acceptable salts, include, but are not limited to acid addition salts, formed by reacting the free base form of the compound with a pharmaceutically acceptable: inorganic acid such as hydrochloric acid, hydrobromic acid, sulfuric acid, nitric acid, phosphoric acid, metaphosphoric acid, and the like; or with an organic acid such as acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfortic acid, 2-hydroxyethanesulfonic acid, benzenesulfonic acid, toluenesulfonic acid, 2-naphthalenesulfonic acid, 4-methytbicyclo-[2.2.2]oct-2-ene-1-carboxylic acid, glucoheptonic acid, 4,4′-methylenebis-(3-hydroxy-2-ene-1-carboxylic acid). 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the chemical structure of geranic acid, or 3,7-dimethyl-2,6-octadienoic acid, is:

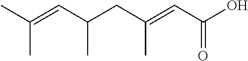

In some embodiments, geranic acid is in the form of a pharmaceutically acceptable salt. The type of pharmaceutical acceptable salts, include, but are not limited to salts formed when an acidic proton present in the parent compound either is replaced by a metal ion, e.g., an alkali metal ion (e.g. lithium, sodium, potassium), an alkaline earth ion (e.g. magnesium, or calcium), or an aluminum ion; or coordinates with an organic base. Examples of acceptable organic bases include, but are not limited to, ethanolamine, diethanolamine, triethanolamine, tromethamine, and N-methylglucamine. Examples of acceptable inorganic bases include, but are not limited to, aluminum hydroxide, calcium hydroxide, potassium hydroxide, sodium carbonate, and sodium hydroxide.

In some embodiments, the choline and the fatty acid are synthesized using any suitable standard synthetic reactions. In some embodiments, the reactions are employed in a linear sequence to provide the compounds or they may be used to synthesize fragments which are subsequently joined by any suitable method. In some embodiments, the starting material used for the synthesis of choline or fatty acid is synthesized or are obtained from commercial sources. In some embodiments, geranic acid is purified from the commercially available technical grade (Sigma-Aldrich, St. Louis, Mo.) by repeated (5-7×) recrystallization from a solution of 70 wt % geranic acid/30 wt % acetone at −70° C. In some embodiments, purity of the geranic acid is assessed by ¹H NMR spectroscopy and conductivity measurements. In some embodiments, the term geranic acid refers to a geranic acid or a salt thereof. In some embodiments, the geranic acid is an anti-microbial agent.

### Topical Composition of RNAi Agents

In some embodiments, each component in a composition, such as the RNAi agents, ionic liquid, the pharmaceutically acceptable carrier, and optionally other components, is described a percent (%) of the composition. In some embodiments, the % of the composition is a percent concentration volume/volume (v/v) or a percent concentration weight/volume (w/v).

Disclosed herein, in certain embodiments, is a topical composition, comprising the RNAi agents or a pharmaceutically acceptable salt thereof, and an ionic liquid in an amount sufficient to allow a therapeutically effective amount of the RNAi agents to reach a target depth within or beyond the skin, wherein the ionic liquid comprises a choline cation and a fatty acid anion.

In some embodiments, the RNAi agents are selected from the group consisting of interfering RNAs (siRNAs), microRNAs (miRNAs), small hairpin RNAs (shRNAs), and combinations thereof. In sonic embodiments, the RNAi agents are selected from the group consisting of siRNA, miRNAs, and combinations thereof. In some embodiments, the RNAi agents are siRNAs.

In some embodiments, the target depth is from about 0.05 mm to about 20 mm. In some embodiments, the target depth is from about 0.1 mm to about 15 mm. In some embodiments, the target depth is from about 1 mm to about 10 mm.

In some embodiments, the pharmaceutical composition allows therapeutically effective amount of the RNAi agents to be delivered into the stratum contemn layer. In some embodiments, the pharmaceutical composition allows therapeutically effective amount of the RNAi agents to be delivered into the epidermis layer. In some embodiments, the pharmaceutical composition allows therapeutically effective amount of the RNAi agents to reach at least a dermis layer. In some embodiments, the pharmaceutical composition allows therapeutically effective amount of the RNAi agents to reach at least the subcutaneous tissue layer. In some embodiments, the pharmaceutical composition allows therapeutically effective amount of the RNAi agents to reach at least the muscle tissue.

In some embodiments, the total amount of the RNAi agents delivered into the stratum corneum layer provided by the pharmaceutical composition is greater than the total amount of the RNAi agents delivered into the stratum corneum layer provided by a control composition without the ionic liquid. In some embodiments, the total amount of the RNAi agents delivered into the stratum corneum layer provided by the pharmaceutical composition is at least 1.2 times the total amount of the RNAi agents delivered into the stratum contemn layer provided by a control composition without the ionic liquid.

In some embodiments, the total amount of the RNAi agents delivered to the epidermis and dermis layers provided by the pharmaceutical composition is more than the total amount of the RNAi agents delivered into the epidermis and dermis layers provided by a control composition without the ionic liquid. In some embodiments, the total amount of the RNAi agents delivered to the epidermis and dermis layers provided by the pharmaceutical composition is at least 1.2 times the total amount of the RNAi agents delivered to the epidermis and dermis layers provided by a control composition without the ionic liquid. In some embodiments, the total amount of the RNAi agents delivered to the epidermis and dermis layers provided by the pharmaceutical composition is at least 1.5 times the total amount of the RNAi agents delivered to the epidermis and dermis layers provided by a control composition without the ionic liquid.

In some embodiments, the total amount of the RNAi agents beyond the dermis layer provided by the pharmaceutical composition is more than the total amount of the RNAi agents delivered beyond the dermis layer provided by a control composition without the ionic liquid. In some embodiments, the total amount of the RNAi agents delivered beyond the dermis layer provided by the pharmaceutical composition is at least 1.5 times the total amount of the RNAi agents delivered beyond the dermis layers provided by a control composition without the ionic liquid. In some embodiments, the total amount of the RNAi agents delivered beyond the dermis layer provided by the pharmaceutical composition is at least 2 times the total amount of the RNAi agents delivered beyond the dermis layers provided by a control composition without the ionic liquid.

In some embodiments, the pharmaceutical composition is administered one or more times a day. In some embodiments, the pharmaceutical composition provides reduced systemic exposure to the RNAi agents as compared to therapeutically effective doses of oral the RNAi agents.

In some embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

In some embodiments, the fatty acid is selected from the group consisting oleic acid, geranic acid, hexanoic acid, and malonic acid, In some embodiments, the fatty acid is geranic acid.

In sonic embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. in some embodiments, the ionic liquid comprises the choline cation and fatty acid anion in a molar ratio of 1:2 of choline cation to fatty acid anion.

In some embodiments, wherein the pharmaceutical composition consists essentially of the RNAi agents and the ionic liquid.

In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 85%. In some embodiments, the ionic liquid is present in the pharmaceutical composition at a concentration of from about 10% to about 90%. In some embodiments, the ionic liquid is present in the pharmaceutical composition at a concentration of from about 20% to about 80%. In some embodiments, the ionic liquid is present in the pharmaceutical composition at a concentration of from about 30% to about 60%. In some embodiments, the ionic liquid is present in the pharmaceutical composition at a concentration of from about 40% to about 60%. In some embodiments, the ionic liquid is present in the pharmaceutical composition at a concentration of about 45% to about 55%. In some embodiments, the ionic liquid is present in the pharmaceutical composition at a concentration of about 50%.

In some embodiments, the pharmaceutical composition further comprises a penetration enhancer. In some embodiments, the penetration enhancer is 2-(2-ethoxyethoxy)ethanol or oleyl alcohol. In some embodiments, the penetration enhancer is 2-(2-ethoxyethoxy)ethanol. In some embodiments, the pharmaceutical composition comprises from about 1% to about 20% of 2-(2-ethoxyethoxy)ethanol. In some embodiments, the pharmaceutical composition comprises from about 5% to about 15% 2-(2-ethoxyethoxy)ethanol. In some embodiments, the pharmaceutical composition comprises from about 10% 2-(2-ethoxyethoxy)ethanol.

In some embodiments, the pharmaceutical composition is essentially free of 2-(2-ethoxyethoxy)ethanol or oleyl alcohol.

In some embodiments, the composition comprises the ionic liquid in a concentration of about 0.1% to 99%. In some embodiments, the composition comprises the ionic liquid in a concentration of about 1% to 40%. In some embodiments, the composition comprises the ionic liquid in a concentration of about 1% to 20%. In some embodiments, the composition comprises the ionic liquid in a concentration of about 5% to 20%. In some embodiments, the composition comprises the ionic liquid in a concentration of about 5% to 40%. In some embodiments, the composition comprises the ionic liquid in a concentration of about 20% to 40%. In some embodiments, the composition comprises the ionic liquid in a concentration of about 20% to 60%. In some embodiments, the composition comprises the ionic liquid in a concentration of about 20% to 80%.

In some embodiments, the composition comprises the ionic liquid in a concentration of about 0.1% to 99%, and the pharmaceutically acceptable solvent in a concentration of about 1% to about 99.9%. In some embodiments, the composition comprises the ionic liquid in a concentration of about 1% to 40%, and the pharmaceutically acceptable solvent in a concentration of about 60% to about 99%. In some embodiments, the composition comprises the ionic liquid in a concentration of about 20% to 40%, and the pharmaceutically acceptable solvent in a concentration of about 80% to about 99%. In some embodiments, the composition comprises the ionic liquid in a concentration of about 20% and the pharmaceutically acceptable solvent in a concentration of about 80%. In some embodiments, the composition comprises the ionic liquid in a concentration of about 40% and the pharmaceutically acceptable solvent in a concentration of about 60%.

In some embodiments, the composition comprises the ionic liquid in a concentration of about I% to 50%, and the pharmaceutically acceptable solvent in a concentration of about 50% to 99%. In sonic embodiments, the composition comprises the ionic liquid in a concentration of about 1% to 50%, and water in a concentration of about 50% to 99%. In some embodiments, the water is deionized water or Milli-Q® water.

In some embodiments, the composition comprises the ionic liquid in a concentration of about 1% to 50%, a pharmaceutically acceptable solvent in a concentration of about 1% to 50%, and a gelling agent in a concentration of about 1 to 5%. In some embodiments, the composition comprises the ionic liquid in a concentration of about 1% to 50%, and water in a concentration of about 1% to 50%.

In some embodiments, the pharmaceutically acceptable solvent is diisopropyl adipate. In some embodiments, the composition comprises diisopropyl adipate in a concentration of about 20%. In some embodiments, the composition comprises the ionic liquid in a concentration of about 1% to 40%, and diisopropyl adipate in a concentration of about 60% to about 99%.

In some embodiments, the composition comprises a gel base in a concentration of about 50% to 90% of the composition. In some embodiments, the composition comprises a gel base in a concentration of about 50%, 60%, 70%, 80%, or 90% of the composition.

In some embodiments, preparing an ionic liquid comprising a choline cation and a fatty acid anion comprises: (a) mixing choline and a fatty acid in a solvent at room temperature in a predetermined ratio; and (b) removing the solvent in vacuo. In some embodiments, the fatty acid is geranic acid. In some embodiments, the solvent is water. In a particular embodiment, the water is deionized water. In some embodiments, removing the solvent comprises rotary evaporation. In some embodiments, removing the solvent comprises heating the ionic liquid, applying a vacuum to the ionic liquid, or a combination thereof. In some embodiments, preparing the ionic liquid further comprises drying the ionic liquid. In some embodiments, heating the ionic liquid comprises heating the ionic liquid to 60° C. In some embodiments, the heating is done for at least 10 minutes, 20 minutes, 30 minutes. 1 hour, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours, In some embodiments, the vacuum is applied at −100 kPa. In some embodiments, the vacuum is applied for at least 10 minutes, 20 minutes, 30 minutes, 1 hours, 2 hours, 3 hours, 4 hours, 5 hours, 6 hours, 7 hours, 8 hours, 9 hours, 10 hours, 11 hours, 12 hours, 24 hours, 36 hours, 48 hours or 60 hours.

In some embodiments, the ionic liquid has had the solvent used in the ionic liquid preparation process removed. In some embodiments, the ionic liquid does not comprise water.

In some embodiments, choline is choline bicarbonate. In some embodiments, the choline is choline in an 80% wt solution of choline bicarbonate. In some embodiment, the predetermined ratio is a ratio of 1:1, 1:2, 1:3, or 1:4 of a choline cation:fatty acid anion. In one embodiment, the ratio is a molar ratio. In another embodiment, the ratio is ratio by weight.

In some embodiments, isolating the composition further comprises purifying the ionic liquid. In some embodiments, purifying the ionic liquid comprises using conventional techniques, including, but not limited to, filtration, distillation, crystallization, and chromatography. In some embodiments, preparing the ionic liquid further comprises isolating the purified ionic liquid

### RNAi Topical Delivery

Disclosed herein, in certain embodiments, is a method of delivering RNA interference (RNAi) agents to or through a skin, the method comprising administering to the skin a pharmaceutical composition comprising the RNAi agents, and an ionic liquid in an amount sufficient to allow a therapeutically effective amount of the RNAi agents to reach a target depth within or beyond the skin, wherein the ionic liquid comprises a choline cation and a fatty acid anion.

In some embodiments, the RNAi agents are selected from the group consisting of interfering RNAs (siRNAs), microRNAs (miRNAs), small hairpin RNAs (shRNAs), and combinations thereof. In some embodiments, the RNAi agents are selected from the group consisting of siRNA, miRNAs, and combinations thereof. In some embodiments, the RNAs agents are siRNAs.

In some embodiments, the target depth is from about 0.05 mm to about 20 mm. In sonic embodiments, the target depth is from about 0.1 mm to about 15 mm. In some embodiments, the target depth is from about 1 mm to about 10 mm.

In some embodiments, the pharmaceutical composition allows therapeutically effective amount of the RNAi agents to be delivered into the stratum corneum layer. In some embodiments, the pharmaceutical composition allows therapeutically effective amount of the RNAi agents to be delivered into the epidermis layer. In some embodiments, the pharmaceutical composition allows therapeutically effective amount of the RNAi agents to reach at least a dermis layer. In some embodiments, the pharmaceutical composition allows therapeutically effective amount of the RNAi agents to reach at least the subcutaneous tissue layer. In some embodiments, the pharmaceutical composition allows therapeutically effective amount of the RNAi agents to reach at least the muscle tissue.

In some embodiments, the total amount of the RNAi agents delivered into the stratum corneum layer provided by the pharmaceutical composition is greater than the total amount of the RNAi agents delivered into the stratum corneum layer provided by a control composition without the ionic liquid. In some embodiments, the total amount of the RNAi agents delivered into the stratum contemn layer provided by the pharmaceutical composition is at least 1.2 times the total amount of the RNAi agents delivered into the stratum corneum layer provided by a control composition without the ionic liquid.

In some embodiments, the total amount of the RNAi agents delivered to the epidermis and dermis layers provided by the pharmaceutical composition is more than the total amount of the RNAi agents delivered into the epidermis and dermis layers provided by a control composition without the ionic liquid. In some embodiments, the total amount of the RNAi agents delivered to the epidermis and dermis layers provided by the pharmaceutical composition is at least 1.2 times the total amount of the RNAi agents delivered to the epidermis and dermis layers provided by a control composition without the ionic liquid. In some embodiments, the total amount of the RNAi agents delivered to the epidermis and dermis layers provided by the pharmaceutical composition is at least 1.5 times the total amount of the RNAi agents delivered to the epidermis and dermis layers provided by a control composition without the ionic liquid.

In some embodiments, the total amount of the RNAi agents beyond the dermis layer provided by the pharmaceutical composition is more than the total amount of the RNAi agents delivered beyond the dermis layer provided by a control composition without the ionic liquid. In some embodiments, the total amount of the RNAi agents delivered beyond the dermis layer provided by the pharmaceutical composition is at least 1.5 times the total amount of the RNAi agents delivered beyond the dermis layers provided by a control composition without the ionic liquid. In some embodiments, the total amount of the RNAi agents delivered beyond the dermis layer provided by the pharmaceutical composition is at least 2 times the total amount of the RNAi agents delivered beyond the dermis layers provided by a control composition without the ionic liquid.

In some embodiments, the pharmaceutical composition is administered one or more times a day. In some embodiments, the pharmaceutical composition provides reduced systemic exposure to the RNAi agents as compared to therapeutically effective doses of oral the RNAi agents.

In sonic embodiments, the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eieosapentaenoic acid, erucic acid, docosahexaenoic acid, propionic acid, butyric acid, valeric acid, hexanoic acid, malonic acid, enanthic acid, caprylic acid, pelaraonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontvlic acid, lacceroic acid, psyllic acid, geddic acid, ceroplastic acid, or hexatriacontylic acid.

In some embodiments, the fatty acid is selected from the group consisting oleic acid, geranic acid, hexanoic acid, and malonic acid. In some embodiments, the fatty acid is geranic acid.

In some embodiments, the ionic liquid is a deep eutectic solvent (DES). In some embodiments, the ionic liquid comprises the choline cation and fatty acid anion in a molar ratio in a range of 1:1 to 1:4 of choline cation to fatty acid anion. In some embodiments, the ionic liquid comprises the choline cation and fatty acid anion in a molar ratio of 1:2 of choline cation to fatty acid anion.

In some embodiments, wherein the pharmaceutical composition consists essentially of the RNAi agents and the ionic liquid.

In some embodiments, the pharmaceutical composition further comprises a pharmaceutically acceptable carrier. In some embodiments, the pharmaceutically acceptable carrier is an aqueous carrier. In some embodiments, the pharmaceutically acceptable carrier comprises an ointment base.

In some embodiments, the ionic liquid is present in the pharmaceutical composition at a concentration of from about 1% to about 99%. In some embodiments, the ionic liquid is present in the pharmaceutical composition at a concentration of from about 5% to about 85%. In some embodiments, the ionic liquid is present in the pharmaceutical composition at a concentration of from about 10% to about 90%. In some embodiments, the ionic liquid is present in the pharmaceutical composition at a concentration of from about 20% to about 80%. In some embodiments, the ionic liquid is present in the pharmaceutical composition at a concentration of from about 30% to about 60%. In some embodiments, the ionic liquid is present in the pharmaceutical composition at a concentration of from about 40% to about 60%. In some embodiments, the ionic liquid is present in the pharmaceutical composition at a concentration of about 45% to about 55%. In some embodiments, the ionic liquid is present in the pharmaceutical composition at a concentration of about 50%.

In some embodiments, the pharmaceutical composition further comprises a penetration enhancer. In some embodiments, the penetration enhancer is 2-(2-ethoxyethoxy)ethanol or oleyl alcohol. In some embodiments, the penetration enhancer is 2-(2-ethoxyethoxy)ethanol. In some embodiments, the pharmaceutical composition comprises from about 1% to about 20% of 2-(2-ethoxyethoxy)ethanol. In some embodiments, the pharmaceutical composition comprises from about 5% to about 15% 2-(2-ethoxyethoxy)ethanol. In some embodiments, the pharmaceutical composition comprises from about 10% 2-(2-ethoxyethoxy)ethanol.

In some embodiments, the pharmaceutical composition is essentially free of 2-(2-ethoxyethoxy)ethanol or oleyl alcohol.

### Certain Terminology

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits arc also included in the methods and compositions described herein.

The terms “individual,” “patient,” or “subject” are used interchangeably. None of the terms require or are limited to situation characterized by the supervision (e.g. constant or intermittent) of a health care worker (e,g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to he prevented. For example, a subject or mammal is successfully “treated” for rosacea, if, after receiving a therapeutic amount of a composition according to the methods of the present disclosure, the subject shows observable and/or measurable reduction in or absence of one or more of the following: reduction in the erythema; reduction in the appearance of red veins; papules, and pustules.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein is an amount effective to achieve a desired pharmacologic effect or therapeutic improvement without undue adverse side effects. It is understood that “an effect amount” or “a therapeutically effective amount” can vary from subject to subject, due to variation in metabolism of the compound, age, weight, general condition of the subject, the condition being treated, the severity of the condition being treated, and the judgment of the prescribing physician. By way of example only, therapeutically effective amounts may be determined by routine experimentation, including but not limited to a dose escalation clinical trial.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Certain Non-Limiting Embodiments

Embodiment 1. A method of delivering RNA interference (RNAi) agents to or through a skin, the method comprising administering to the skin a pharmaceutical composition comprising the RNAi agents, and an ionic liquid in an amount sufficient to allow a therapeutically effective amount of the RNAi agents to reach a target depth within or beyond the skin, wherein the ionic liquid comprises a choline cation and a fatty acid anion.
Embodiment 2. The method of Embodiment 1, wherein the RNAi agents are selected from the group consisting of interfering RNAs (siRNAs), microRNAs (miRNAs), small hairpin RNAs (shRNAs), and combinations thereof.
Embodiment 3. The method of Embodiment 1, wherein the RNAi agents are selected from the group consisting of siRNA, miRNAs, and combinations thereof.
Embodiment 4. The method of Embodiment 1, wherein the RNAi agents are siRNAs,
Embodiment 5. The method of any one of Embodiment 1-4, wherein the target depth is from about 0.05 mm to about 20 mm.
Embodiment 6. The method of any one of Embodiment 1-4. wherein the target depth is from about 0,1 mm to about 15 mm.
Embodiment 7. The method of any one of Embodiment 1-4, wherein the target depth is from about 1 rum to about 10 mm.
Embodiment 8. The method of any one of Embodiment 1-7, wherein the pharmaceutical composition allows therapeutically effective amount of the RNAi agents to reach at least an epidermis layer.
Embodiment 9. The method of Embodiment 8, wherein the amount of the RNAi agents at the epidermis layer is sufficient to reduce expression or translation of at least one target gene within the epidermis layer.
Embodiment 10. The method of any one of Embodiment 1-9, wherein the pharmaceutical composition allows therapeutically effective amount of the RNAi agents to reach at least a dermis layer.
Embodiment 11. The method of Embodiment 10, wherein the amount of the RNAi agents at the dermis layer is sufficient to reduce expression or translation of at least one target gene within the dermis layer.
Embodiment 12. The method of any one of Embodiment 1-11, wherein the pharmaceutical composition allows therapeutically effective amount of the RNAi agents to reach at least the a subcutaneous tissue layer.
Embodiment 13. The method of Embodiment 12, wherein the amount of the RNAi agents at the subcutaneous tissue layer is sufficient to reduce expression or translation of at least one target gene within the subcutaneous tissue layer.
Embodiment 14. The method of any one of Embodiment 1-13, wherein the pharmaceutical composition allows therapeutically effective amount of the RNAi agents to reach at least a muscle tissue.
Embodiment 15. The method of Embodiment 14, wherein the amount of the. RNAi agents at the muscle tissue is sufficient to reduce expression or translation of at least one target gene within the muscle tissue.
Embodiment 16, The method of any one of Embodiment 1-15, wherein the total amount of the RNAi agents delivered into the stratum corneum layer provided by the pharmaceutical composition is greater than the total amount of the RNAi agents delivered into the stratum corneum layer provided by a control composition without the ionic liquid.
Embodiment 17. The method of any one of Embodiment 1-15. wherein the total amount of the RNAi agents delivered into the stratum corneum layer provided by the pharmaceutical composition is at least 1.2 times the total amount of the RNAi agents delivered into the stratum corneum layer provided by a control composition without the ionic liquid.
Embodiment 18. The method of any one of Embodiment 1-17, wherein the total amount of the RNAi agents delivered to the epidermis and dermis layers provided by the pharmaceutical composition is more than the total amount of the RNAi agents delivered into the epidermis and. dermis layers provided by a control composition without the ionic liquid.
Embodiment 19. The method of any one of Embodiment 1-17, wherein the total amount of the RNAi agents delivered to the epidermis and dermis layers provided by the pharmaceutical composition is at least 1.2 times the total amount of the RNAi agents delivered to the epidermis and dermis layers provided by a control composition without the ionic liquid.
Embodiment 20. The method of any one of Embodiment 1-17, wherein the total amount of the RNAi agents delivered to the epidermis and dermis layers provided by the pharmaceutical composition is at least 1.5 times the total amount of the RNAi agents delivered to the epidermis and dermis layers provided by a control composition without the ionic liquid.
Embodiment 21. The method of any one of Embodiment 1-20, wherein the total amount of the RNAi agents beyond the dermis layer provided by the pharmaceutical composition is more than the total amount of the RNAi agents delivered beyond the dermis layer provided by a control composition without the ionic liquid.
Embodiment 22. The method of any one of Embodiment 1-20, wherein the total amount of the RNAi agents delivered beyond the dermis layer provided by the pharmaceutical composition is at least 1.5 times the total amount of the RNAi agents delivered beyond the dermis layers provided by a control composition without the ionic liquid.
Embodiment 23. The method of any one of Embodiment 1-20, wherein the total amount of the RNAi agents delivered beyond the dermis layer provided by the pharmaceutical composition is at least 2 times the total amount of the RNAi agents delivered beyond the dermis layers provided by a control composition without the ionic liquid.
Embodiment 24. The method of any one of Embodiment 1-23, wherein the pharmaceutical composition is administered one or more times a day.
Embodiment 25. The method of any one of Embodiment 1-24, wherein the pharmaceutical composition provides reduced systemic exposure to the RNAi agents as compared to therapeutically effective doses of oral the RNAi agents.
Embodiment 26. The method of any one of Embodiment 1-25, wherein the fatty acid is selected from the group consisting of myristoleic acid, palmitoleic acid, sapienic acid, oleic acid, elaidic acid, geranic acid, vaccenic acid, linoleic acid, linoelaidic acid. α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.
Embodiment 27. The method of any one of Embodiment 1-25, wherein the fatty acid is selected from the group consisting oleic acid, geranic acid, hexanoic acid, and malonic acid.
Embodiment 28. The method of any one of Embodiment 1-25, wherein the fatty acid is geranic acid.
Embodiment 29. The method of any one of Embodiment 1-28, wherein the ionic liquid is a deep eutectic solvent (DES).
Embodiment 30. The method of Embodiment 1-29. wherein the ionic liquid comprises the choline cation and fatty acid anion in a molar ratio in a range of 1:1 to 1:4 of choline cation to fatty acid anion.
Embodiment 31. The method of Embodiment 1-29, wherein the ionic liquid comprises the choline cation and fatty acid anion in a molar ratio of 1:2 of choline cation to fatty acid anion.
Embodiment 32. The method of Embodiment 1-30, wherein the pharmaceutical composition consists essentially of the RNAi agents and the ionic liquid.
Embodiment 33. The method of Embodiment 1-30, wherein the pharmaceutical composition further comprises a pharmaceutically acceptable carrier.
Embodiment 34. The method of Embodiment 33, wherein the pharmaceutically acceptable carrier is an aqueous carrier.
Embodiment 35. The method of Embodiment 33. wherein the pharmaceutically acceptable carrier comprises an ointment base.
Embodiment 36. The method of Embodiment 1-35, wherein the ionic liquid is present in the pharmaceutical composition at a concentration of from about 1% to about 99%.
Embodiment 37. The method of Embodiment 1-35. wherein the ionic liquid is present in the pharmaceutical composition at a concentration of from about 5% to about 85%.
Embodiment 38. The method of Embodiment 1-35, wherein the ionic liquid is present in the pharmaceutical composition at a concentration of from about 10% to about 90%.
Embodiment 39. The method of Embodiment 1-35, wherein the ionic liquid is present in the pharmaceutical composition at a concentration of from about 20% to about 80%.
Embodiment 40. The method of Embodiment 1-35, wherein the ionic liquid is present in the pharmaceutical composition at a concentration of from about 30% to about 60%.
Embodiment 41. The method of Embodiment 1-35, wherein the ionic liquid is present in the pharmaceutical composition at a concentration of from about 40% to about 60%.
Embodiment 42. The method of Embodiment 1-35, wherein the ionic liquid is present in the pharmaceutical composition at a concentration of about 45% to about 55%.
Embodiment 43. The method of Embodiment 1-35, wherein the ionic liquid is present in the pharmaceutical composition at a concentration of about 50%.
Embodiment 44. The method of Embodiment 1-43. wherein the pharmaceutical composition further comprises a penetration enhancer.
Embodiment 45. The method of Embodiment 44, wherein the penetration enhancer is 2-(2-ethoxyethoxy)ethanol or oleyl alcohol.
Embodiment 46. The method of Embodiment 44. wherein the penetration enhancer is 2-(2-ethoxyethoxy)ethanol.
Embodiment 47. The method of Embodiment 46, wherein the pharmaceutical composition. comprises from about 1% to about 20% of 2-(2-ethoxyethoxy)ethanol.
Embodiment 48. The method of Embodiment 46, wherein the pharmaceutical composition comprises from about 5% to about 15% 2-(2-ethoxyethoxy)ethanol.
Embodiment 49. The method of Embodiment 46. wherein the pharmaceutical composition comprises from about 10% 2-(2-ethoxyethoxy)ethanol.
Embodiment 50. The method of Embodiment 1-43, wherein the pharmaceutical composition is essentially free of 2-(2-ethoxyethoxy)ethanol or oleyl alcohol,
Embodiment 51. A topical composition, comprising the RNAi agents or a pharmaceutically acceptable salt thereof, and an ionic liquid in an amount sufficient to allow a therapeutically effective amount of the RNAi agents to reach a target depth within or beyond the skin, wherein the ionic liquid comprises a choline cation and a fatty acid anion.
Embodiment 52. The composition of Embodiment 51, wherein the RNAi agents are selected from the group consisting of interfering RNAs (siRNAs), microRNAs (miRNAs), small hairpin RNAs (shRNAs), and combinations thereof.
Embodiment 53. The composition of Embodiment 51, wherein the RNAi agents are selected from the group consisting of siRNA, miRNAs, and combinations thereof.
Embodiment 54. The composition of Embodiment 51, wherein the RNAi agents are siRNAs.
Embodiment 55. The composition of any one of Embodiment 51-54, wherein the target depth is from about 0.05 min to about 20 mm.
Embodiment 56. The composition of any one of Embodiment 51-54, wherein the target depth is from about 0.1 mm to about 15 mm.
Embodiment 57. The composition of any one of Embodiment 51-54, wherein the target depth is from about 1 mm to about 10 mm.
Embodiment 58. The composition of any one of Embodiment 51-57, wherein the pharmaceutical composition allows therapeutically effective amount of the RNAi agents to be delivered into the stratum corneum layer.
Embodiment 59. The composition of any one of Embodiment 51-58, wherein the pharmaceutical composition allows therapeutically effective amount of the RNAi agents to be delivered into the epidermis layer.
Embodiment 60. The composition of any one of Embodiment 51-59, wherein the pharmaceutical composition allows therapeutically effective amount of the RNAi agents to reach at least a dermis layer.
Embodiment 61. The composition of any one of Embodiment 51-60, wherein the pharmaceutical composition allows therapeutically effective amount of the RNAi agents to reach at least the subcutaneous tissue layer.
Embodiment 62. The composition of any one of Embodiment 51-61, wherein the pharmaceutical composition allows therapeutically effective amount of the RNAi agents to reach at least the muscle tissue.
Embodiment 63. The composition of any one of Embodiment 51-62, wherein the total amount of the RNAi agents delivered into the stratum corneum layer provided by the pharmaceutical composition is greater than the total amount of the RNAi agents delivered into the stratum corneum layer provided by a control composition without the ionic liquid.
Embodiment 64. The composition of any one of Embodiment 51-62, wherein the total amount of the RNAi agents delivered into the stratum corneum layer provided by the pharmaceutical composition is at least 1.2 times the total amount of the RNAi agents delivered into the stratum corneum layer provided by a control composition without the ionic liquid.
Embodiment 65. The composition of any one of Embodiment 51-64, wherein the total amount of the RNA agents delivered to the epidermis and dermis layers provided by the pharmaceutical composition is more than the total amount of the RNAi agents delivered into the epidermis and dermis layers provided by a control composition without the ionic liquid.
Embodiment 66. The composition of any one of Embodiment 51-64, wherein the total amount of the RNAi agents delivered to the epidermis and dermis layers provided by the pharmaceutical composition is at least 1.2 times the total amount of the RNAi agents delivered to the epidermis and dermis layers provided by a control composition without the ionic liquid.
Embodiment 67. The composition of any one of Embodiment 51-64, wherein the total amount of the RNAi agents delivered to the epidermis and dermis layers provided by the pharmaceutical composition is at least 1.5 times the total amount of the RNAi agents delivered to the epidermis and dermis layers provided by a control composition without the ionic liquid.
Embodiment 68. The composition of any one of Embodiment 51-67, wherein the total amount of the RNAi agents beyond the dermis layer provided by the pharmaceutical composition is more than the total amount of the RNAi agents delivered beyond the dermis layer provided by a control composition without the ionic liquid.
Embodiment 69. The composition of any one of Embodiment 51-67, wherein the total amount of the RNAi agents delivered beyond the dermis layer provided by the pharmaceutical composition is at least 1.5 times the total amount of the RNAi agents delivered beyond the dermis layers provided by a control composition without the ionic liquid.
Embodiment 70. The composition of any one of Embodiment 51-67, wherein the total amount of the RNAi agents delivered beyond the dermis layer provided by the pharmaceutical composition is at least 2 times the total amount of the RNAi agents delivered beyond the dermis layers provided by a control composition without the ionic liquid.
Embodiment 71. The composition of any one of Embodiment 51-70, wherein the pharmaceutical composition is administered one or more times a day.
Embodiment 72. The composition of any one of Embodiment 51-71, wherein the pharmaceutical composition provides reduced systemic exposure to the RNAi agents as compared to therapeutically effective doses of oral the RNAi agents.
Embodiment 73. The composition of any one of Embodiment 51-72, wherein the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid. eicosapentaenoic acid, erucic acid, docosahexaenoic acid, propionic acid, butyric acid, valeric acid, hexanoic acid, tnalonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.
Embodiment 74. The composition of any one of Embodiment 51-72, wherein the fatty acid is selected from the group consisting oleic acid, geranic acid, hexanoic acid, and malonic acid.
Embodiment 75. The composition of any one of Embodiment 51-72, wherein the fatty acid is geranic acid.
Embodiment 76. The composition of any one of Embodiment 51-75, wherein the ionic liquid is a deep eutectic solvent (DES).
Embodiment 77. The composition of Embodiment 51-76, wherein the ionic liquid comprises the choline cation and fatty acid anion in a molar ratio in a range of 1:1 to 1:4 of choline cation to fatty acid anion.
Embodiment 78. The composition of Embodiment 51-76, wherein the ionic liquid comprises the choline cation and fatty acid anion in a molar ratio of 1:2 of choline cation to fatty acid anion.
Embodiment 79. The composition of Embodiment 51-78, wherein the pharmaceutical composition consists essentially of the RNAi agents and the ionic liquid.
Embodiment 80. The composition of Embodiment 51-79, wherein the pharmaceutical composition further comprises a pharmaceutically acceptable carrier.
Embodiment 81. The composition of Embodiment 80, wherein the pharmaceutically acceptable carrier is an aqueous carrier.
Embodiment 82. The composition of Embodiment 80, wherein the pharmaceutically acceptable carrier comprises an ointment base.
Embodiment 83. The composition of Embodiment 51-82, wherein the ionic liquid is present in the pharmaceutical composition at a concentration of from about 1% to about 99%.
Embodiment 84. The composition of Embodiment 51-82, wherein the ionic liquid is present in the pharmaceutical composition at a concentration of from about 5% to about 85%.
Embodiment 85. The composition of Embodiment 51-82, wherein the ionic liquid is present in the pharmaceutical composition at a concentration of from about 10% to about 90%.
Embodiment 86. The composition of Embodiment 51-82, wherein the ionic liquid is present in the pharmaceutical composition at a concentration of from about 20% to about 80%.
Embodiment 87. The composition of Embodiment 51-82, wherein the ionic liquid is present in the pharmaceutical composition at a concentration of from about 30% to about 60%.
Embodiment 88. The composition of Embodiment 51-82, wherein the ionic liquid is present in the pharmaceutical composition at a concentration of from about 40% to about 60%,
Embodiment 89. The composition of Embodiment 51-82, wherein the ionic liquid is present in the pharmaceutical composition at a concentration of about 45% to about 55%.
Embodiment 90. The composition of Embodiment 51-82, wherein the ionic liquid is present in the pharmaceutical composition at a concentration of about 50%.
Embodiment 91. The composition of Embodiment 51-82, wherein the pharmaceutical composition further comprises a penetration enhancer.
Embodiment 92. The composition of Embodiment 91, wherein the penetration enhancer is 2-(2-ethoxyethoxy)ethanol or oleyl alcohol.
Embodiment 93. The composition of Embodiment 91, wherein the penetration enhancer is 2-(2-ethoxyethoxy)ethanol.
Embodiment 94. The composition of Embodiment 93, wherein the pharmaceutical composition comprises from about 1% to about 20% of 2-(2-ethoxyethoxy)ethanol.
Embodiment 95. The composition of Embodiment 93, wherein the pharmaceutical composition comprises from about 5% to about 15% 2-(2-ethoxyethoxy)ethanol.
Embodiment 96. The composition of Embodiment 93, wherein the pharmaceutical composition comprises from about 10% 2-(2-ethoxyethoxy)ethanol.
Embodiment 97. The composition of Embodiment 51-90, wherein the pharmaceutical composition is essentially free of 2-(2-ethoxyethoxy)ethanol or oleyl alcohol.

In some embodiments the topical the RNAi agents composition described herein provides improved stability or less degradation of the RNAi agents therein. In some embodiments, the topical the RNAi agents composition described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 1% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to the RNAi agents degradation over a period of at least about 1 week. Also described herein are formulations that are stable with respect to the RNAi agents degradation over a period of at least about 1 month.

### NON-LIMITING EXAMPLES

### Example 1: Preparation of an Ionic Liquid Containing Choline Cation and Geranic Acid Anion in a 1:1 Molar Ratio (Ionic Liquid B)

The purified GMP Penta Geranic acid (311.0 g, 1.848 mol) was placed in a 2 L round bottomed flask. The flask was placed in a water bath at 20° C. and stirred. Then choline bicarbonate (381.7 g, 1.848 mol) 80% solution in water (Sigma, 07519, 209 ml) was added slowly (drop-wise) with an addition funnel, total addition time was 120 min. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −1.00 kPa to remove the resulting water. After no more water evaporation was observed by condensation on the dry ice trap of the rotavap, the flask was further heated at 60° C. and −100 kPa for 36 additional hrs to dry the final product. 475 g of product (94.7% yield) was obtained. HPLC analysis shows 97.9% purity.

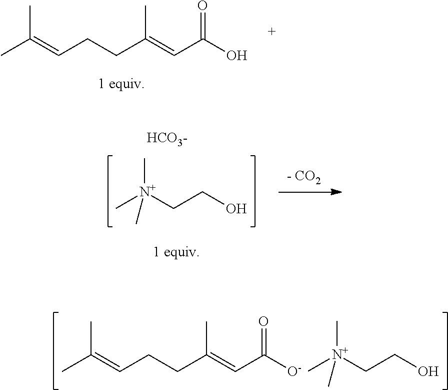

### Example 2: Preparation of an Ionic Liquid Containing Choline Cation and Geranic Acid Anion in a 1:2 Molar Ratio (Ionic Liquid A)

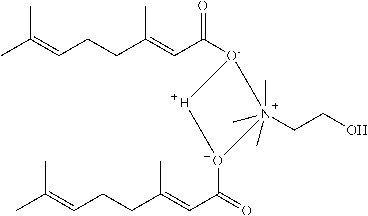

To two equivalents (9.88 g., 0.059 moles) of neat geranic acid, recrystallized 5× at −70° C. from 70% geranic acid/30% acetone, in a 500 mL round bottom flask was added one equivalent of choline bicarbonate (80 wt % solution, 6.06 g, 0029 mol). The mixture was stirred at room temperature until no more CO₂ evolved. Solvent was removed by rotary evaporation at 60° C. for 20 min, and the product was dried in a vacuum oven for 48 h at 60° C.

Physical characterization at 25° C.: solubility in water=0.5 M; density=0.990 g/mL; conductivity=0.0431 mS/cm; viscosity=1345 cP.

### Example 3: Alternate Preparation of an Ionic Liquid Containing Choline Cation and Geranic Acid Anion in a 1:2 Molar Ratio (Ionic Liquid A)

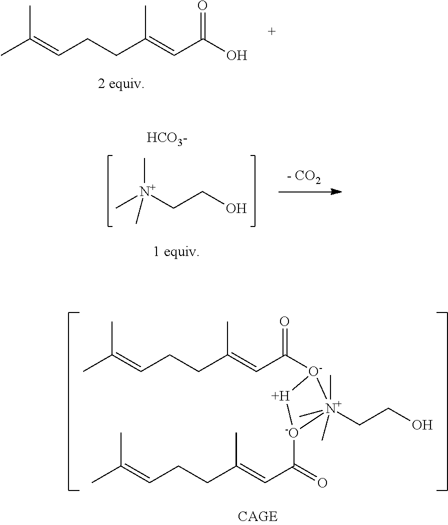

The purified GMP Penta Geranic acid (155 g, 0.921 mol) was placed in a 1 L round bottomed flask. The flask was placed in a water bath at 20° C. and stirred. Then choline bicarbonate (95.1 g, 0.460) 80% solution in water (Sigma, C7519, Lot #: 059K1526V, 209 ml) was added slowly (drop-wise) with an addition funnel, total addition time was 35 chin. The flask was stirred overnight (12 hrs) to maximize the escape of the resulting CO₂. The flask was placed in the rotavap and the remaining CO₂ was removed at room temperature (20° C.) and a small vacuum (30 mbar). After no more CO₂ evolution was observed in the form of foam, the bath was heated to 60° C. and vacuum increased to −100 kPa to remove the resulting water. After no more water evaporation was observed by condensation on the dry ice trap of the rotavap, the flask was further heated at 60° C. and −100kPa for 36 additional hrs to dry the final product. 197 g of Cage (96% yield) was obtained. 1H-NMR spectrum looks similar to the one of CB-0001. HPLC analysis shows 95.1% purity.

### Example 4: Preparation of an Ionic Liquid Containing Choline Cation and Geranic Acid a 1:3 Molar Ratio

To three equivalents (14.56 g., 0.087 moles) of neat geranic acid, recrystallized 5× at −70° C. from 70% geranic acid/30% acetone, in a 1000 mL round bottom flask is added one equivalent of choline bicarbonate (80 wt. % solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 5: Preparation of an Ionic Liquid Containing Choline and Geranic Acid in a 1:4 Molar Ratio

To four equivalents (19.76 g., 0.118 moles) of neat geranic acid, recrystallized 5× at −70° C. from 70% geranic acid/30% acetone, in a 800 mL round bottom flask is added one equivalent of choline bicarbonate (80 wt solution, 6.06 g, 0.029 mol). The mixture is stirred at room temperature until no more CO₂ evolved. Solvent is removed by rotary evaporation at 60° C. for 20 min, and the product is dried in a vacuum oven for 48 h at 60° C.

### Example 6: Preparation of a Topical Composition Containing the RNAi Agents and Ionic Liquid

An siRNA, Ionic Liquid A (according to Example 2) and RNase-free water are mixture together until a homogenous composition is achieved.

TABLE 1

| Components | Concentration |
|---|---|
| siRNA | 1-500 μM |
| Ionic Liquid A | 10-90% (w/w) |
| RNase-free water | balance |

### Example 7: Preparation of a Topical Composition Containing the RNAi Agents and Ionic Liquid

An siRNA, Ionic Liquid A (according to Example 2) and RNase-free water are mixture together until a homogenous composition is achieved.

TABLE 2

| Components | Concentration |
|---|---|
| siRNA | 1-500 μM |
| Ionic Liquid A | 30-70% (w/w) |
| RNase-free water | balance |

### Example 8: Preparation of a Topical Composition Containing the RNAi Agents and Ionic Liquid

An miRNA, Ionic Liquid A (according to Example 2) and RNase-free water are mixture together until a homogenous composition is achieved.

TABLE 3

| Components | Concentration |
|---|---|
| miRNA | 1-500 μM |
| Ionic Liquid A | 10-90% (w/w) |
| RNase-free water | balance |

### Example 9: Preparation of a Topical Composition Containing the RNAi Agents and Ionic Liquid

An miRNA, Ionic Liquid A (according to Example 2) and RNase-free water are mixture together until a homogenous composition is achieved.

TABLE 4

| Components | Concentration |
|---|---|
| miRNA | 1-500 μM |
| Ionic Liquid A | 30-70% (w/w) |
| RNase-free water | balance |

### Example 10: Preparation of a Topical Composition Containing the RNAi Agents and Ionic Liquid

An shRNA, Ionic Liquid A (according to Example 2) and RNase-free water are mixture together until a homogenous composition is achieved.

TABLE 5

| Components | Concentration |
|---|---|
| shRNA | 1-500 μM |
| ionic Liquid A | 10-90% (w/w) |
| RNase-free water | balance |

### Example 11: Preparation of a Topical Composition Containing the RNAi Agents and Ionic Liquid

An shRNA, Ionic Liquid A (according to Example 2) and RNase-free water are mixture together until a homogenous composition is achieved.

TABLE 6

| Components | Concentration |
|---|---|
| shRNA | 1-500 μM |
| Ionic Liquid A | 30-70% (w/w) |
| RNase-free water | balance |

### Example 12: Preparation of a Topical Composition Containing the RNAi Agents and Ionic Liquid (RNAi Composition A)

An siRNA, Ionic Liquid A (according to Example 2) and RNase-free water are mixture together until a homogenous composition is achieved. The sequence of the siRNA is Cy5.G.A.C.G.U.A.A.A.C.G.G.C.C.A.C.A.A.G.U.U.C.U.U.

TABLE 7
RNAi Composition A

| Components | Concentration |
|---|---|
| siRNA | 50 μM |
| Ionic Liquid A | 50% (w/w) |
| RNase-free water | balance |

### Example 13: Preparation of a Topical Composition Containing the RNA Agents, Ionic Liquid, and Transcutol® (RNAi Composition B)

The same siRNA, Ionic Liquid A (according to Example 2), 2-(2-ethoxyethoxy)ethanol (Transcutol®), and RNase-free water are mixture together until a homogenous composition is achieved.

TABLE 8
RNAi Composition B

| Components | Concentration |
|---|---|
| siRNA | 50 μM |
| Ionic Liquid A | 50% (w/w) |
| Transcutol ® | 10% (w/w) |
| RNase-free water | balance |

### Example 14: Preparation of a Topical Composition Containing the RNAi Agents (Control Composition A)

The same siRNA and RNase-free water are mixture together until a homogenous composition is achieved.

TABLE 9
Control Composition A

| Components | Concentration |
|---|---|
| siRNA | 50 μM |
| RNase-free water | balance |

### Example 15: Skin Flux Assay of the RNAi Agents Compositions

The RNAi agents delivery by the following four compositions was measured using a static Franz cell setup (N=3 for each composition).

1. RNAi Composition A (N=3)

2. Control Composition A (N==3)

Study was performed in Franz cells with porcine skin clamped over receptor chamber containing saline. The test composition (10 μL) was applied to surface of skin and occluded. After 24 h at 37° C., the skin sample was tape stripped 10 times to evaluate the distribution of siRNA delivered to the stratum corneum (SC2-SC10). The first strip (SC1) corresponds to the siRNA remaining on the surface of the stratum corneum. The tape stripping sample was then extracted in methanol:saline, 1:1, v/v. Dermis and epidermis layers were extracted together and not separated (“D/E”). Receptor is the saline underneath the skin that was sampled directly. The concentration of the siRNA in these samples was quantified by HPLC.

Cumulative skin flux of the RNAi agents was shown in FIG. 1. The data demonstrated that compositions containing ionic liquid according to non-limiting embodiments of the present disclosure resulted in a significantly increased delivery of the siRNA in the stratum corneum, epidermis and dermis layers, as well as to deeper tissues (e.g. subcutaneous tissues and muscle), compared to the control composition that does not contain the ionic liquid.

### Example 16: Non-Irritating to the Skin

The compositions described herein are typically non-irritating to the skin. Each of the components in the IL (i,e,, anionic and cationic components) may on its own be irritating to the skin. However, the combination of the ionic components used in the complex that is included in the composition is not irritating, or substantially non-irritating (i.e. causes at most a minimal skin reaction) when applied to the surface of the skin.

The compositions may cause minimal or no skin reaction, such as redness, rash, inching, burning or tingling sensations. Minimal skin reaction may be understood as slight skin reaction with signs of irritation but one that is not uncomfortable or painful to the subject.

Typically, the compositions are non-toxic to the skin cells. The compositions do not induce significant adverse reactions in the healthy skin cells, such as reduction in viability of healthy skin cell, when applied to the skin.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A topical composition, comprising one or more RNAi agents or a pharmaceutically acceptable salt thereof, and an ionic liquid in an amount sufficient to allow a therapeutically effective amount of the RNAi agents to reach a target depth within or beyond the skin, wherein the ionic liquid comprises a choline cation and a fatty acid anion.

2. The topical composition of claim 1, wherein the RNAi agents are selected from the group consisting of interfering RNAs (siRNAs), microRNAs (miRNAs), small hairpin RNAs (shRNAs), and combinations thereof.

3. The topical composition of claim 1, wherein the RNAi agents are selected from the group consisting of siRNA, miRNAs, and combinations thereof.

4. The topical composition of claim 1, wherein the RNAi agents are siRNAs.

5. The topical composition of claim 1, wherein the fatty acid is selected from the group consisting of myristoleic acid, palmitoleic acid, sapienic acid, oleic acid, elaidic acid, geranic acid, vaccenic acid, linoleic acid, linoelaidic acid, α-linolenic acid, arachidonic acid, eicosapentaenoic acid, erucic acid, docosahexaenoic acid, propionic acid, butyric acid, valeric acid, hexanoic acid, malonic acid, enanthic acid, caprylic acid, pelargonic acid, capric acid, undecylic acid, lauric acid, tridecyclic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, or hexatriacontylic acid.

6. The topical composition of claim 1, wherein the fatty acid is selected from the group consisting oleic acid, geranic acid, hexanoic acid, and malonic acid.

7. The topical composition of claim 1, wherein the fatty acid is geranic acid.

8. The topical composition of claim 1, wherein the ionic liquid is a deep eutectic solvent (DES).

9. The topical composition of claim 1, wherein the ionic liquid comprises the choline cation and fatty acid anion in a molar ratio in a range of 1:1 to 1:4 of choline cation to fatty acid anion.

10. The topical composition of claim 1, wherein the ionic liquid comprises the choline cation and fatty acid anion in a molar ratio of 1:2 of choline cation to fatty acid anion.

11. The topical composition of claim 1, wherein the pharmaceutical composition consists essentially of the RNAi agents and the ionic liquid.

12. The topical composition of claim 1, wherein the pharmaceutical composition further comprises a pharmaceutically acceptable carrier.

13. The topical composition of claim 12, wherein the pharmaceutically acceptable carrier is an aqueous carrier.

14. The topical composition of claim 12, wherein the pharmaceutically acceptable carrier comprises an ointment base.

15. The topical composition of claim 1, wherein the ionic liquid is present in the pharmaceutical composition at a concentration of from about 1% to about 99%.

16. The topical composition of claim 1, wherein the ionic liquid is present in the pharmaceutical composition at a concentration of from about 5% to about 85%.

17. The topical composition of claim 1, wherein the ionic liquid is present in the pharmaceutical composition at a concentration of from about 10% to about 90%.

18. The topical composition of claim 1, wherein the ionic liquid is present in the pharmaceutical composition at a concentration of from about 20% to about 80%.

19. The topical composition of claim 1, wherein the ionic liquid is present in the pharmaceutical composition at a concentration of from about 30% to about 60%.

20. The topical composition of claim 1, wherein the ionic liquid is present in the pharmaceutical composition at a concentration of from about 40% to about 60%.

21. The topical composition of claim 1, wherein the ionic liquid is present in the pharmaceutical composition at a concentration of about 45% to about 55%.

22. The topical composition of claim 1, wherein the ionic liquid is present in the pharmaceutical composition at a concentration of about 50%.

23. The topical composition of claim 1, wherein the pharmaceutical composition is essentially free of any additional penetration enhancer.

24. The topical composition of claim 1, wherein the target depth is from about 0.05 mm to about 20 mm.

25. The topical composition of claim 1, wherein the target depth is from about 1 mm to about 10 mm.

26. The topical composition of claim 1, wherein the pharmaceutical composition allows therapeutically effective amount of the RNAi agents to be delivered into the stratum corneum layer.

27. The topical composition of any one of claims 26, wherein the total amount of the RNAi agents delivered into the stratum corneum layer provided by the pharmaceutical composition is greater than the total amount of the RNAi agents delivered into a stratum corneum layer provided by a control composition without the ionic liquid.

28. The topical composition of claim 1, wherein the pharmaceutical composition allows therapeutically effective amount of the RNAi agents to be delivered into at least one of epidermis and dermis layer.

29. The topical composition of claim 28, wherein the total amount of the RNAi agents delivered to the at least one of epidermis and dermis layers provided by the pharmaceutical composition is more than the total amount of the RNAi agents delivered into at least one of epidermis and dermis layers provided by a control composition without the ionic liquid.

30. The topical composition of claim 1, wherein the pharmaceutical composition allows therapeutically effective amount of the RNAi agents to reach beyond a dermis layer.

31. The topical composition of claim 30, wherein the total amount of the RNAi agents beyond the dermis layer provided by the pharmaceutical composition is more than the total amount of the RNAi agents delivered beyond a dermis layer provided by a control composition without the ionic liquid.
